(19) United States
(12) Patent Application Publication
(10) Pub. No.: US 2024/0027465 A1
(43) Pub. Date: Jan. 25, 2024

(54) METABOLIC AUGMENTATION TO PROMOTE AND ENHANCE IMMUNE RESPONSE BY TCF1+ T CELL REPOPULATION

(71) Applicants: Steven L. REINER, NewYork, NY (US); Brian S. HENICK, New York, NY (US); Naiyer A. RIZVI, New York, NY (US); Rohan MANIAR, New York, NY (US); The Trustees of Columbia University in the City of New York, New York, NY (US)

(72) Inventors: Steven L Reiner, Mount Kisco, NY (US); Brian S Henick, Englewood, NJ (US); Naiyer A Rizvi, New York, NY (US); Rohan Maniar, West Windsor, NJ (US)

(73) Assignee: The Trustees of Columbia University in the City of New York, New York, NY (US)

(21) Appl. No.: 18/245,797

(22) PCT Filed: Sep. 23, 2021
(86) PCT No.: PCT/US2021/051805

§ 371 Date: Mar. 17, 2023

Related U.S. Application Data
(60) Provisional application No. 63/232,549, filed on Aug. 12, 2021.
(60) Provisional application No. 63/082,238, filed on Sep. 23, 2020.

(51) Int. Cl. G01N 33/68; A61K 31/52; A61P 37/02

(52) U.S. Cl. CPC G01N 33/68; A61K 31/52; A61P 37/02; G01N 2800/52

(57) ABSTRACT

A method of treating a condition in a subject by improving the immune response of the subject comprising first determining the level of TCF1 in the subject to identify the subject as having an anti-PD-1 responder phenotype or an anti-PD-1 non-responder phenotype, then administering an anti-PD-1 treatment to a subject having an anti-PD-1 responder phenotype or a metabolic inhibitor prior to ant-PD-1 treatment to a subject having an anti-PD-1 non-responder phenotype.

This invention was made with government support under grant numbers AI076458, AI113365, and AI061699 awarded by the National Institutes of Health (NIH). The government has certain rights in the invention.

Throughout this application, various publications are referenced, including referenced in parenthesis. The disclosures of all publications mentioned in this application in their entireties are hereby incorporated by reference into this application in order to provide additional description of the art to which this invention pertains and of the features in the art which can be employed with this invention.

### TECHNICAL FIELDS

This application relates to a method of improving an immune response in a subject. The method may be used to treat a condition of a subject, including conditions such as chronic infectious disease, autoimmune disorders, and cancer. The method may also be used to monitor the clinical response of a subject and treat the subject accordingly.

### BACKGROUND OF THE INVENTION

Immune checkpoint inhibitors are an increasingly utilized type of immunotherapy for treating several conditions, including cancer. However, checkpoint inhibitor immunotherapy options currently have a limited duration of success and vary in effectiveness from patient to patient. Identification of markers that indicate whether a patient will benefit from such checkpoint inhibitor immunotherapies will help improve patient selection. Additionally, new treatment options for patients that are unlikely to benefit from such immune checkpoint inhibitor therapies alone will further improve patient outcomes.

Previously, PI3K signaling has been linked to T cell differentiation and silencing of TCF1 expression (Lin et al., 2015). It has also been shown that CD8⁺ TCF1⁺ cells self-renew in vivo (Lin et al. 2016) and that TCF1 may be utilized as a marker of self-renewal of human T cells (Kratchmarov et al., 2018). Furthermore, it has been demonstrated showed that PI3K represents anabolic cell metabolism generally, expanding targets to other anabolism-associated pathways. It has also been shown that in vitro cells undergoing over-differentiation and loss of self-renewal might be rescued with anti-anabolic strategies (Adams et al., 2016).

TCF1 has also been shown as a marker of self-renewal of CD4⁺ T cells (Nish et al. 2017) and that PI3K signaling directs the lop-sided divisions of T cells allowing the stem cell behavior of simultaneous self-renewal and differentiation (Chen et al. 2018). Other groups pointed out that TCF1⁺ marks progenitors during chronic infection and cancer (Im et al, 2016); Utzschneider et al., 2016; Wu et al., 2016; and Philip et al, 2017) and that a proliferative T cell burst following checkpoint blockade correlates to favorable therapeutic response to checkpoint blockade (Huang et al., 2017; Huang et al., 2019; Kamphorst et al., 2017). Intratumoral ratio of TCF1⁺ to TCF1⁻ CD8⁺ T cells has been shown as a predictor of response to checkpoint blockade, and treatments such as CD39 inhibitors have been proposed to improve patient outcome (Sade-Feldman et al., 2018).

Importantly, other studies have pointed to the importance of the TCF1⁺ progenitor but lack any connection to PI3K inhibition as a way to preserve and expand TCF1⁺ T cells. Instead, many studies attempt to develop methods to convert TCF1⁻ to TCF1⁺ cells, which has not been fully productive. See, for example, Blank et al., 2019. There is no mention that anti-PD-1 might accelerate loss of self-renewal. Accordingly, a better strategy may be to buffer the preservation of TCF1⁺ cells before they are lost. Inhibition of PI3K improved outcome of checkpoint blockade in a tumor model, but was not connected to TCF1⁺ T cell preservation (Lu et al., 2017) and inhibition of PI3K in cellular expansion protocol of adoptive cell therapy improved outcome, but also was not connected to TCF1⁺ T cell preservation (Bowers et al., 2017).

### SUMMARY OF THE INVENTION

Provided herein is an approach to treat a condition in a subject by promotion and enhancement of the immune response of the subject. The immune response of the subject may be determined by the level of TCF1⁺ expression in the subject. Furthermore, modulating repopulation of the TCF1⁺ T cell population of the subject may be achieved by administration of an anti-anabolic agent. Accordingly, the approaches provided herein are suitable for prevention, monitoring, and treatment of a condition in a subject. For example, the methods provided herein are suitable for tailoring an anti-PD-1 therapy to a subject by additional administration of a metabolic inhibitor, preferably an anti-anabolic agent.

According to an embodiment of the present invention, there is provided a method of treating a condition in a subject by improving the immune response of the subject, the method comprising first determining the level of TCF1 in the subject to identify the subject as having an anti-PD-1 responder phenotype or an anti-PD-1 non-responder phenotype; and second administering an anti-PD-1 treatment to a subject having an anti-PD-1 responder phenotype, or administering a metabolic inhibitor to a subject having an anti-PD-1 non-responder phenotype.

According to another embodiment of the present invention, there is provided a method of treating a condition in a subject by providing an anti-PD-1 treatment to the subject, comprising the steps of first administering an anti-PD1 treatment to the subject, followed by monitoring the clinical response of the condition in the subject to the anti-PD-1 therapy, then identifying reduced anti-PD-1 treatment efficacy in the subject and classifying the subject as having an anti-PD-1 non-responder phenotype if the clinical response decreases, and finally administering a metabolic inhibitor to a subject identified as having an anti-PD-1 non-responder phenotype.

### BRIEF DESCRIPTION OF THE DRAWINGS

FIGS. 1A-1C: Tracking antigen-specific responses serially from tumor aspirates. Tumor size (FIG. 1A) and flow cytometry (FIG. 1B) analyses of serial fine-needle aspirates of Day 14 and Day 19 tumors of mice challenged with MCA205-Ova fibrosarcoma either with (square) or without (circle) anti-PD-1 therapy. Numbers in plots indicate percentages (%). Control mice had progressive tumor growth, with stable infiltration of tumoral CD8⁺, tetramer⁺, TCF1⁺ cells, albeit insufficient for tumor control. αPD-1-treated mice had initial tumor control with brisk CD8⁺, tetramer⁺ infiltrate but a predictably smaller TCF1⁺ fraction. Prior to eventual tumor progression, αPD-1 mouse lost tumoral CD8⁺ pool, with a scant TCF1⁺ fraction. Accordingly, one mode of acquired resistance to a PD-1 blockade may be accelerated loss of self-renewing progenitors.

FIGS. 2A-2B: Serial, non-invasive blood immune monitoring of αPD-1 responses. Tumor size (FIG. 2A) and flow cytometry (FIG. 2B) analyses of weekly blood samples of mice challenged with MC38 colon carcinoma either with (triangle & square) or without (circle) αPD-1 therapy. Numbers in plots indicate percentages (%). Only CD8⁺, CD44^{hi} events are displayed. Control lacked characteristic early burst of TCF1⁻, Ki-67⁺ associated with αPD-1 (Control (8%) compared to αPD-1 treated (23% & 35%). αPD-1 treated mouse with primary resistance (early progressive tumor growth; triangle, middle row of FIG. 2B) failed to maintain TCF1⁺, Ki-67⁺ population compared to αPD-1 mouse with good clinical response (tumor control; square, lower row of FIG. 2B). Not shown: αPD-1 mice with late progression (acquired resistance) typically exhibit loss of TCF1⁺, Ki-67⁺ population prior to tumor progression.

FIGS. 3A-3D: PI3Kδ inhibition (iPI3Kδ) added to αPD-1 improves CD8⁺ T cell durability and TCF1⁺ frequency compared to αPD-1 alone. Numbers in plots indicate percentages (%). FIG. 3A: Inhibiting PI3Kδ in vitro prevents excess division and blocks TCF1 silencing. FIG. 3B: Inhibiting PI3Kδ in vivo increases intratumoral TCF1⁺, Tim3⁻, CD8⁺ T cell abundance in mice with MCA205-Ova tumors. FIG. 3C: Intratumoral infiltration of gated tetramer⁺ T cells in mice challenged with B16F10-Ova melanoma tumors suggests T cell persistence or renewal improves with combination treatment. FIG. 3D: In mice with MC38 tumors, TCF1⁺, Tim3⁻, CD8⁺ T cell loss associated with αPD-1 therapy, might be partly reversed by addition of iPI3Kδ.

FIGS. 4A-4B: iPI3Kδ added to αPD-1 improves control of tumor growth of MCA205-Ova fibrosarcoma (FIG. 4A) and MC38 colon carcinoma (FIG. 4B). Representative tumor growth for four treatment groups.

FIGS. 5A-5B: Regenerative T cell indices in αPD-1 versus αPD-1+iPI3Kδ-treated mice challenged with MC38 tumors. FIG. 5A: FACS of CD8⁺, CD44^{hi} events in serial blood samples from mice treated with αPD-1 (square) or αPD-1+iPI3Kδ combination (triangle) revealed stability of TCF1⁺, Ki67⁺ population and tumor control in combination-treated versus αPD-1 mice. FIG. 5B: Intratumoral CD8⁺ cells revealed better preservation of progenitor TCF1⁺, CD39⁻ population and tumor control in combination-treated animals. These samples are post-mortem but could be detected ante-mortem with fine-needle aspirate.

FIGS. 6A-6B: Schematic representation showing PD-1 regulation of regenerative balance. Forward arrows represent activation signaling. White cells represent precursor cells, light gray cells represent progenitor TCF1⁺ cells, dark gray cells represent differentiated TCF1⁻ cells, black cells represent dysfunctional cells. Normally, TCF1⁺ cells self-renew (looped-back arrows) because inhibitory signals, such as PD-1, support unequal activation of daughter cells (FIG. 6A). Efficiency and uniformity of activation resulting from PD-1 blockade drives new pools of fresh TCF1⁻ cells, but seemingly at the expense of self-renewal (FIG. 6B). The assays presented here aim to predict whether a subject is a suitable candidate for checkpoint blockade based on where in the spectrum between these two responses the subject falls. If the subject is closer to the over-differentiation side, they can be treated with checkpoint blockade if anti-anabolic agents are added.

FIG. 7: Schematic representation of treatment effects on T cells. Compared to no-treatment, αPD-1 intensification of differentiation may have some benefit but its effect on durability makes it limited. The population balance of increased differentiation alongside increased self-renewal offered by combination therapy provides better-than-additive benefit.

FIG. 8: Patient peripheral blood flow cytometry to establish biomarkers of responsiveness and resistance to immune checkpoint blockade. Left, Compiled baseline frequency of TCF1⁺, CCR7⁻, CD8⁺ T cells from 18 non-small cell lung cancer patients (10 Good responders (“Good”); 8 Poor responders (“Poor”)). TCF1⁺ frequency may predict response or resistance to PD-1 blockade (**P=0.0076). Right, Compiled on-treatment ratio of TCF1⁺/TCF1⁻ among PD-1⁺, CCR7⁻, CD8⁺ T cells from 16 NSCLC patient samples (9 Good; 7 Poor). The ratio of TCF1⁺/TCF1⁻ among PD-1⁺, CCR7⁻, CD8⁺ T cells may also be a predictive biomarker to monitor treatment non-invasively (**P=0.0013).

FIGS. 9A-9F: Peripheral blood flow cytometry to establish biomarkers of responsiveness and resistance to Immune Checkpoint Blockade. FIG. 9A: Gating strategy to identify activated CCR7⁻, CD8⁺ T cells (non-naïve, non-central memory). Top-to-bottom, the plots are: lymphocyte gate; CD3⁺ T cell gate; CD8⁺ T cell gate; CCR7⁻, CD8⁺ T cell gate. FIG. 9B: Representative data from four (4) non-small cell lung cancer patients with good response (left two columns) and four (4) with poor response (right two columns) to PD-1 blockade. In each column pair, left column is baseline, right is on-treatment (˜6 weeks after starting immune checkpoint blockade (ICB)). Each row within each column pair is a different patient. FIG. 9C: Compiled baseline frequency of TCF1⁺, CCR7⁻, CD8⁺ T cells from 18 NSCLC patient samples run so far (10 Good responders; 8 Poor responders). TCF1⁺ frequency may predict response or resistance to PD-1 blockade (**P=0.0076). FIG. 9D: Compiled on-treatment ratio of TCF1⁺/TCF1⁻ among PD-1⁺, CCR7⁻, CD8⁺ T cells from 16 NSCLC patient samples run so far (9 Good; 7 Poor). The ratio of TCF1⁺/TCF1⁻ among PD-1⁺, CCR7⁻, CD8⁺ T cells may also be a predictive biomarker to monitor treatment non-invasively (**P=0.0013). FIG. 9E: Analysis of head-and-neck cancer patient with good response to PD-1 blockade, which resembles the good-responder lung cancer patients, thereby suggesting this test will be useful for patients with many types of cancer. FIG. 9F: Healthy blood donor analysis demonstrating a low frequency of PD-1⁺ T cells.

FIG. 10: Inhibitor of PI3K-delta (iPI3Kd) added to anti-PD-1 (αPD-1) might improve control of tumor growth in mice. Representative MC38 carcinoma tumor growth for four (4) indicated treatment groups of mice. Left plot, values are mean measurements of each group, consisting of ≥10 animals per group. Statistical comparison made between combination therapy and the other treatment arms. Right plots represent tumor growth of individual mice in the four (4) groups.

FIG. 11: Anti-PD-1 treatment is associated with loss of TCF1⁺ CD8⁺ T cells in tumor-challenged mice. Ante-mortem blood flow cytometric analysis of MC38 tumor-inoculated mice was performed two weeks after tumor challenge, gating on proliferating CD8⁺ CD44^{hi} T cells. Ratio of TCF1⁺ to TCF1⁻ among proliferating CD8⁺ T cells were quantified from at least four (4) mice per group (control-treated or anti-PD-1-treated), *p=0.019

FIG. 12: Addition of inhibitor of PI3K-delta (iPI3Kd) to augment TCF1⁺ CD8 T cell pool in blood correlates with enhanced efficacy of anti-PD-1 (αPD-1) in mouse cancer model. Blood flow cytometric analysis of MC38 tumor-inoculated mice was performed one week after tumor challenge, gating on proliferating CD8⁺ CD44^{hi} T cells. Ratio of TCF1⁺ to TCF1⁻ among proliferating CD8⁺ T cells were quantified from at least three (3) mice per group one week after tumor challenge, *p=0.0152. Stabilization of TCF1⁺/TCF1⁻ ratio by the addition of iPI3Kd is basis for the proposed use of metabolic manipulation to overcome clinical resistance to anti-PD-1 in patients.

FIGS. 13A-13B: Validating quantitative multiplex immunofluorescence microscopy of human cancer tissue. Markers (with indicated color in FIG. 13B) include CD8, TCF1, CD39, DAPI (DNA dye), and tumor cytokeratin (CK, a lung cancer marker). Two representative tissue cores (FIGS. 13A and 13B) revealing some areas of denser inflammation at the tumor interface containing CD8⁺ TCF1⁺ cells, CD8⁺ TCF1⁻ cells, and some CD39⁺ cells (both TCF1⁺ and TCF1⁻). There are also scattered CD8⁺ TCF1⁻ cells within tumor tissue.

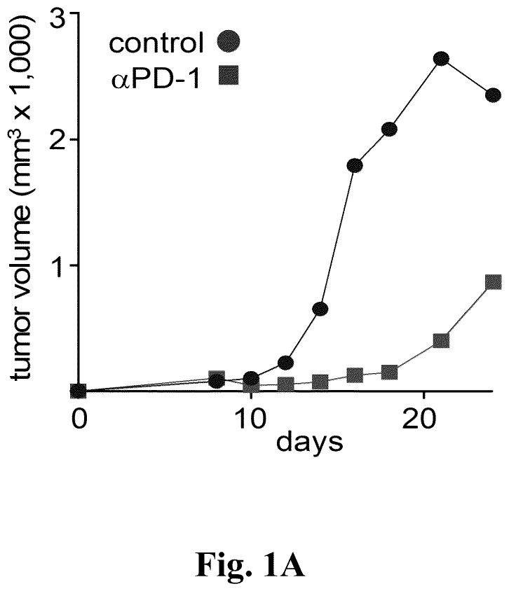

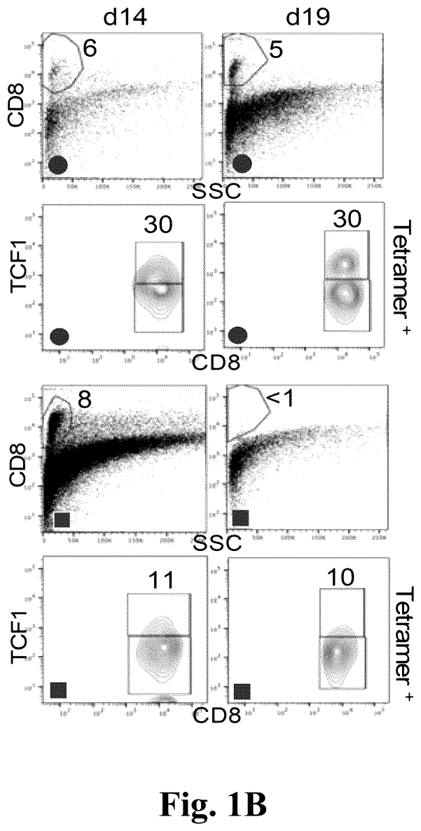

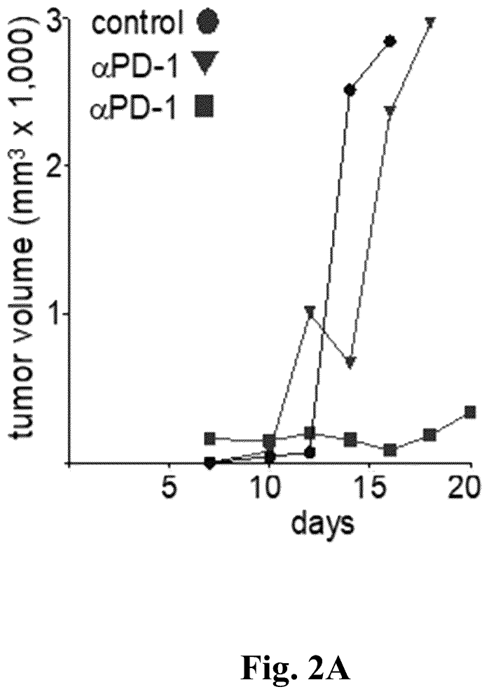

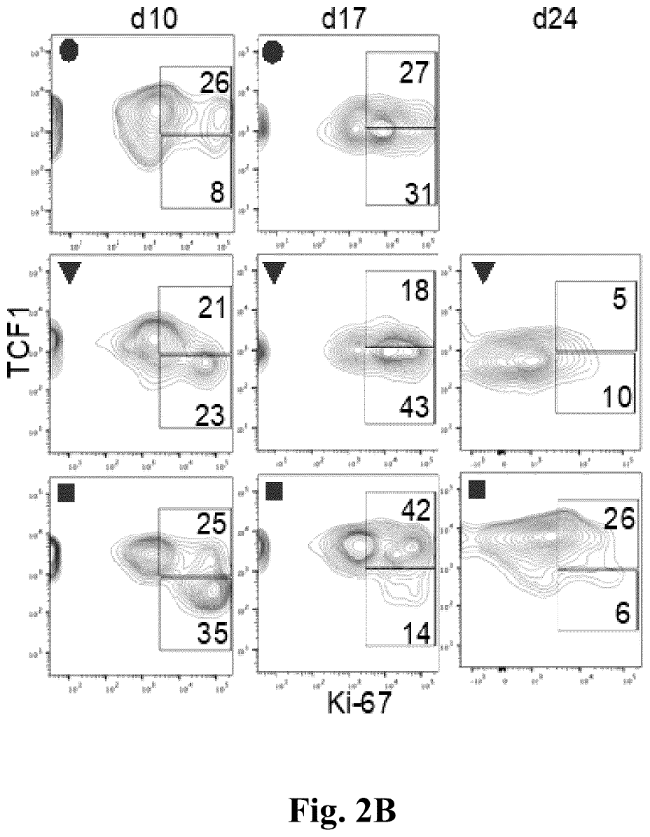

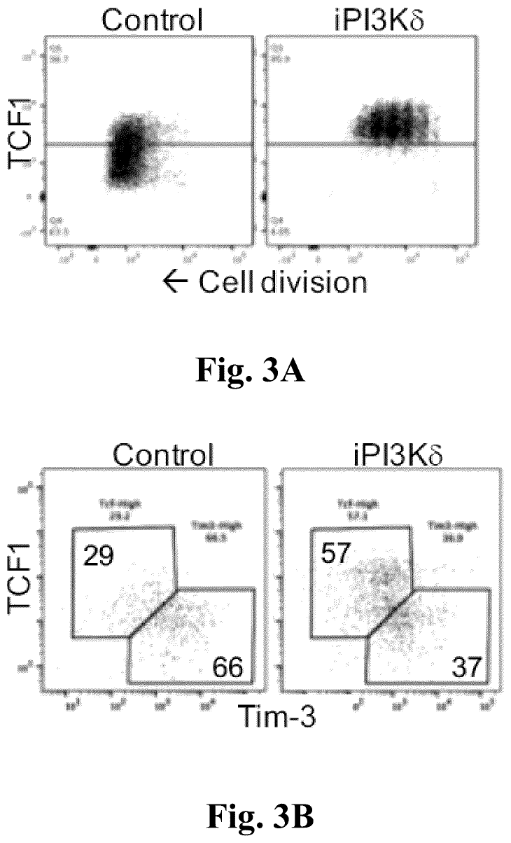

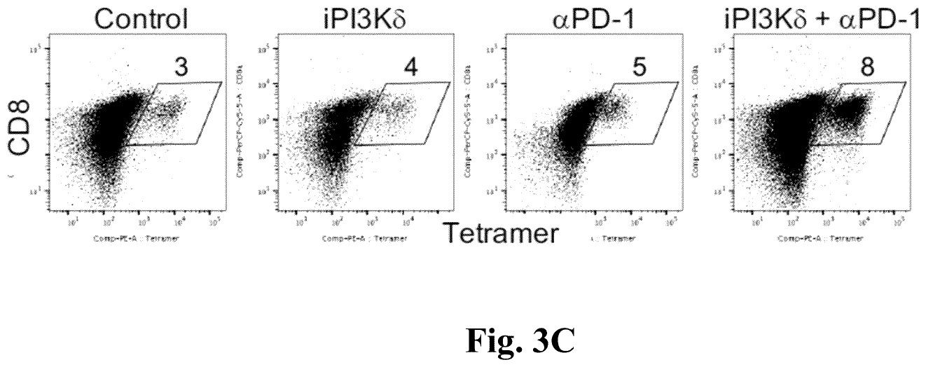

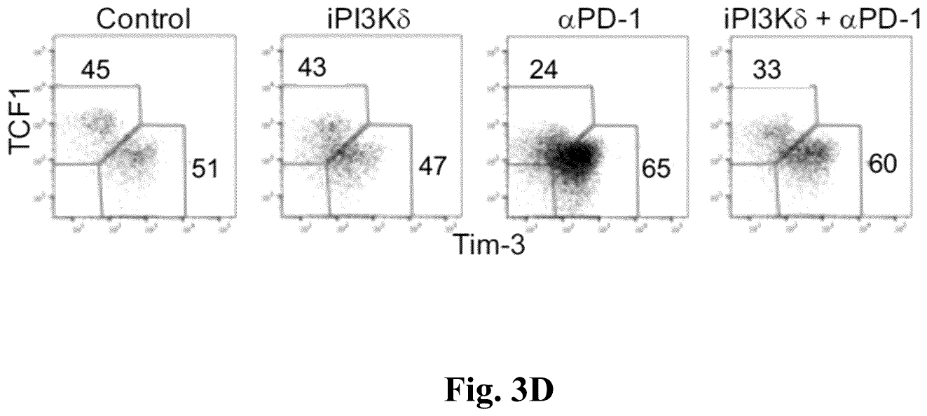

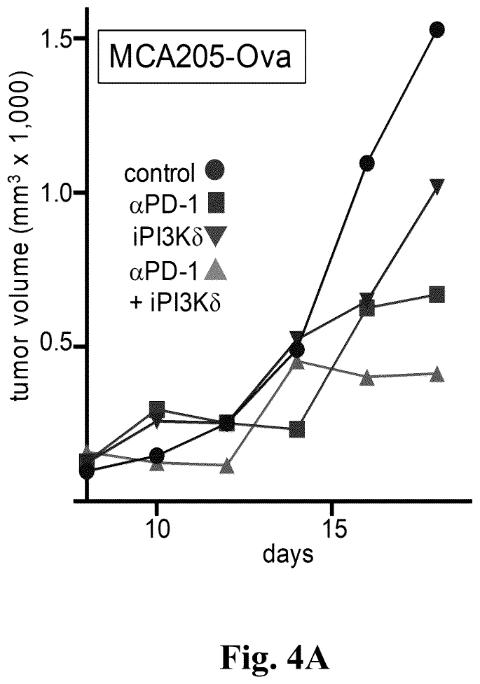

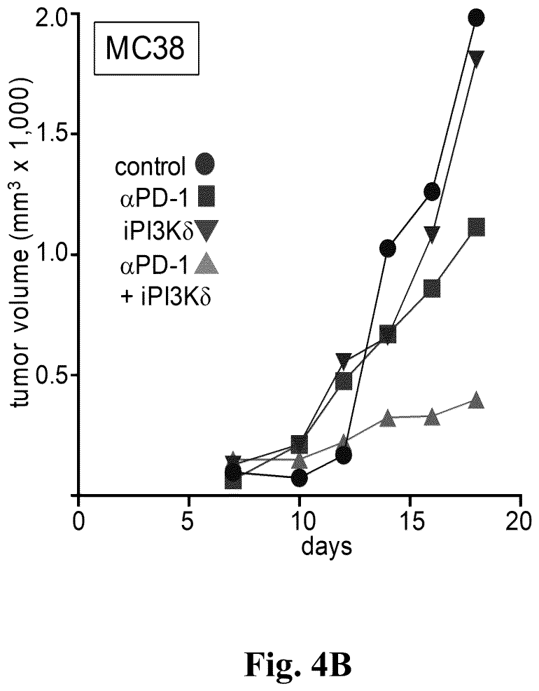

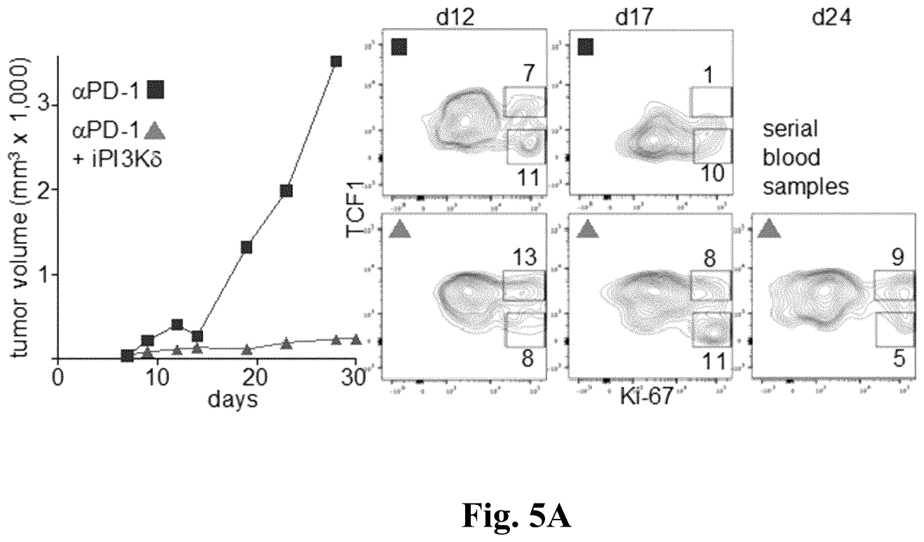

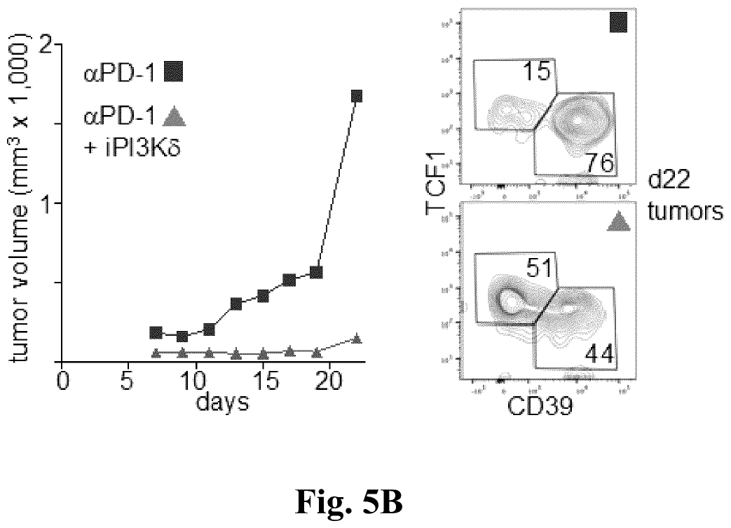

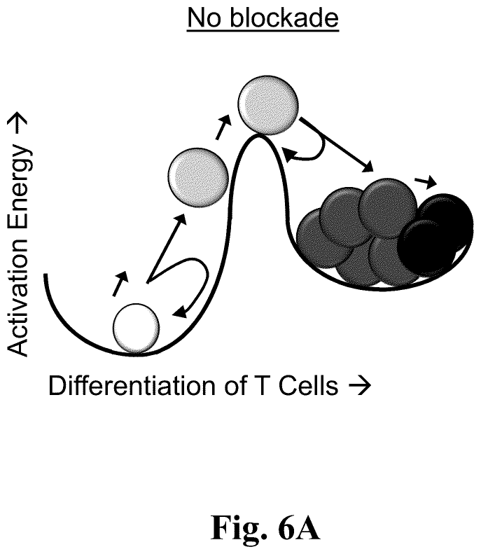

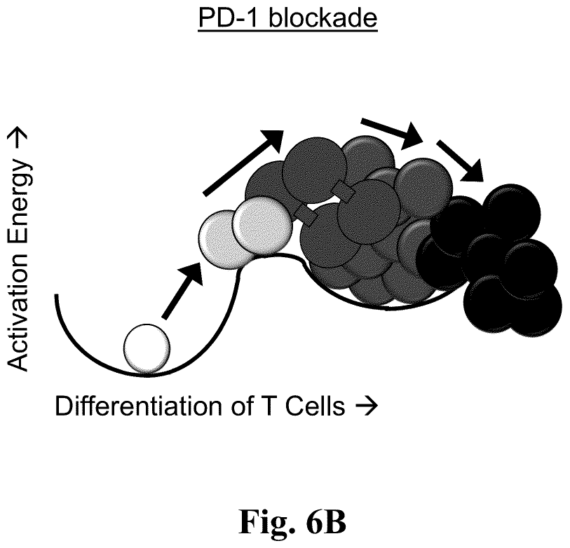

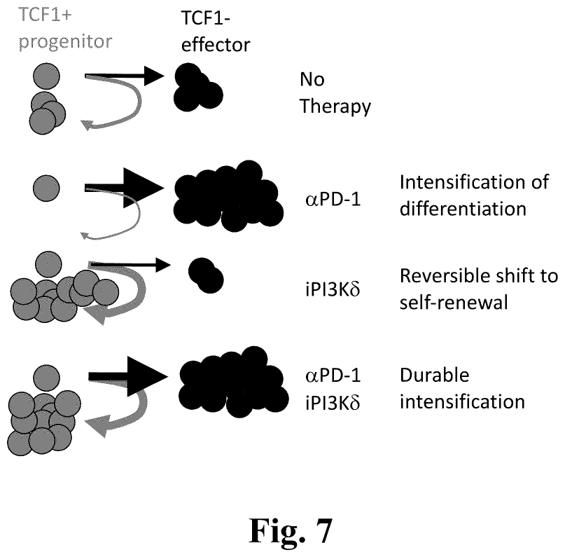

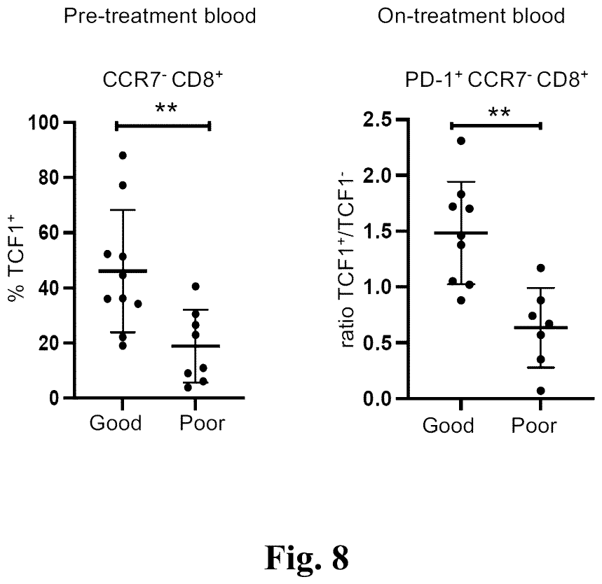

### DETAILED DESCRIPTION

In order to facilitate an understanding of the subject matter disclosed herein, each of the following terms, as used herein, shall have the meaning set forth below, except as expressly provided otherwise herein.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

Unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

In the description and claims of the present application, each of the verbs, “comprise,” “include” and “have” and conjugates thereof, are used to indicate that the object or objects of the verb are not necessarily a complete listing of components, elements or parts of the subject or subjects of the verb. Other terms as used herein are meant to be defined by their well-known meanings in the art.

As used herein, “biomarker” shall mean a measurable indicator of a condition, phenotype, or physiological state of a subject or sample.

As used herein, an “antigen⁺” or “antigen positive” cell (e.g. “TCF1⁺” or “TCF1 positive”) shall mean a cell that has a substantial, readily detectable expression level of the antigen when using routine methods. For example, a cell displaying an expression level of TCF1 that is reasonably above background detection levels is considered TCF1⁺.

An “antigen⁻” or “antigen-negative” cell shall mean a cell that has little to no detectable expression level of the antigen when using routine methods. For example, a cell displaying a level of TCF1 that is not significantly above background detection levels is considered TCF1⁻. Antigen⁺ cells can be readily distinguished from antigen⁻ cells in a sample by measuring the relative expression of the antigen using known techniques, including but not limited to, immunoassays such as flow cytometry, preferably clinical-grade flow cytometry, immunostaining microscopy and immunohistochemistry (IHC) assays. Generally, the average antigen expression level in a population of antigen⁺ cells is at least a ten-fold higher compared to the average antigen expression level in a population of antigen⁻ cells. Generally, antigen⁺ cell counts cluster in a group having an antigen-specific signal well-above antigen⁻ cell counts. Staining controls (e.g. without antibody) also delineate TCF1⁻ from TCF1⁺ cells.

As used herein, “expression level” shall mean any measured level of expression of a gene (e.g. TCF1, Ki-67, CD8, etc.) in a sample. The expression level may be displayed as the number of cells expressing a gene or the amount of expression measured in a gross sample. The level of expression may be determined at the RNA or protein levels. For example, the level of TCF1 expression in a sample may be determined by quantitative RT-PCR, ELISA-based assays, or any other known method in the art. In another specific example, the expression level of TCF1 may be determined by the number of cells expressing a TCF1 above a baseline threshold value e.g. quantifying the number of cells in a tumor sample that are TCF1 positive via clinical-grade flow cytometry.

As used herein, “anti-PD-1 responder phenotype” and “anti-PD-1 non-responder phenotype” shall mean a phenotype that describes the ability of a condition in a subject or sample to improve upon administration of anti-PD-1 therapy. Improvement of a condition in the subject can be determined by a number of factors, including but not limited to a decrease in the severity of symptoms associated with the condition. As a non-limiting example, a tumor having an anti-PD-1 non-responder phenotype will not fully benefit from administration of anti-PD-1 therapy alone and may increase, for example, in size or invasiveness. As disclosed herein, a subject having an anti-PD-1 non-responder phenotype benefits from administration of a metabolic inhibitor with an anti-PD-1 therapy. By increasing the population of TCF1⁺ T cells in a subject, the metabolic inhibitor may alter the phenotype of a subject from an anti-PD-1 non-responder phenotype to an anti-PD-1 responder phenotype.

An anti-PD-1 responder or anti-PD-1 non-responder phenotype can be determined based on, for example, the number of TCF1⁺ T cells in a subject. For example, a subject having a ratio of TCF1⁺/TCF1⁻ cells of about 0.8 or lower is considered to be an anti-PD-1 non-responder. Such a ratio can be determined, for example, using clinical-grade flow cytometry or other methods known in the art. Furthermore, the determination of an anti-PD-1 responder or anti-PD-1 non-responder may be refined using artificial intelligence or machine learning methods. For example, a supervised, unsupervised, or reinforced learning computer program may be used to determine the anti-PD-1 responder or anti-PD-1 non-responder phenotype of a subject. For example, such a method may comprise accessing at one or more computing devices the TCF1 expression level of a test subject, then determining whether the subject has an anti-PD-1 responder phenotype or an anti-PD-1 non-responder phenotype based on a classification engine stored in one or more memories of the one or more computing devices. The classification engine may comprise, for example, a neural network trained on a dataset containing a plurality of subjects' TCF1 expression levels and their associated clinical status over time or clinical outcome after receiving anti-PD-1 therapy.

As used herein, “metabolic inhibitor” shall mean any compound or substance that inhibits a metabolic process, preferably a proliferative or anabolic pathway, in a cell.

As used herein, all numerical ranges provided are intended to expressly include at least the endpoints and all numbers that fall between the endpoints of ranges.

The subject matter disclosed herein enables an enhanced immune response in a subject by promoting TCF1⁺ T cell repopulation. Specifically, TCF1⁺ T cells are increased by inhibiting anabolic metabolism pathways in the subject. These methods are useful for determining if a subject is likely to respond to a therapy, e.g. anti-PD-1 therapy, based on their TCF1 levels, and treating the subject accordingly. These methods are also useful for treating and monitoring several conditions, including but not limited to chronic infectious diseases, autoimmune disorders, and cancer.

The following embodiments and examples (including details thereof) are set forth to aid in an understanding of the subject matter of this disclosure but are not intended to, and should not be construed to, limit in any way the invention that is claimed.

According to embodiments of the present invention, there is provided a method of treating a condition in a subject by improving the immune response of the subject, the method comprising:
a) determining the level of TCF1 in the subject to identify the subject as having an anti-PD-1 responder phenotype or an anti-PD-1 non-responder phenotype; and
b) administering (i) an anti-PD-1 treatment to a subject having an anti-PD-1 responder phenotype; or (ii) a metabolic inhibitor to a subject having an anti-PD-1 non-responder phenotype.

In some embodiments, the level of TCF1 is determined by calculating the proportion of TCF1⁺ T cells among T cells of the subject.

In some embodiments, the level of TCF1 is determined by calculating the proportion of TCF1⁺ T cells among CD8⁺ T cells of the subject.

In some embodiments, the level of TCF1 is determined by calculating the proportion of TCF1⁺ T cells among CD44⁺ T cells of the subject.

In some embodiments, the level of TCF1 is determined by calculating the proportion of TCF1⁺ T cells among Ki-67⁺ T cells of the subject.

In some embodiments, the subject is identified as having an anti-PD-1 non-responder phenotype if the subject displays a ratio of TCF1⁺ T cells/TCF1⁻ T cells of about 0.8 or lower. For example, a subject having less than about 44% of TCF1⁻ positive T cells among all T-cells measured for TCF1 expression levels is identified as having an anti-PD-1 non-responder phenotype.

In some embodiments, the method further comprises determining the level of Ki-67 in the subject to identify the subject as having an anti-PD-1 responder phenotype or an anti-PD-1 non-responder phenotype.

In some embodiments, the level of Ki-67 is determined by calculating the proportion of Ki-67⁺ T cells among T cells of the subject.

In some embodiments, the level of Ki-67 is determined by calculating the proportion of Ki-67⁺ T cells among CD8⁺ T cells of the subject.

In some embodiments, the level of Ki-67 is determined by calculating the proportion of Ki-67⁺ T cells among CD44⁺ T cells of the subject.

In some embodiments, the subject is identified as having an anti-PD-1 non-responder phenotype if the subject displays a ratio of Ki-67⁺ T cells/Ki-67⁻ T cells of about 0.35.

In some embodiments, the method further comprises determining the tumor volume in the subject to identify the subject as having an anti-PD-1 responder phenotype or an anti-PD-1 non-responder phenotype.

In some embodiments, the level of TCF1 is determined by an immunoassay.

In some embodiments, the immunoassay is a flow cytometry assay or an immunohistochemistry assay.

In some embodiments, the administered amount of the metabolic inhibitor is sufficient to increase the level of TCF1 in the subject.

In some embodiments, an anti-PD-1 treatment is administered to the subject identified as having an anti-PD-1 non-responder phenotype simultaneously or substantially simultaneously as the metabolic inhibitor.

In some embodiments, an anti-PD-1 treatment is administered to the subject identified as having an anti-PD-1 non-responder phenotype after detection of an increase in level of TCF1 in the subject.

In some embodiments, the metabolic inhibitor is administered to the subject having an anti-PD-1 responder phenotype.

In some embodiments, the metabolic inhibitor is administered to the subject having an anti-PD-1 responder phenotype simultaneously or substantially simultaneously as the anti-PD-1 treatment.

In some embodiments, the metabolic inhibitor is administered to the subject having an anti-PD-1 responder phenotype after the anti-PD-1 treatment.

In some embodiments, the metabolic inhibitor is an inhibitor of proliferative anabolic metabolism. In some embodiments, the metabolic inhibitor inhibits a proliferative process in a cell. In some embodiments, the metabolic inhibitor inhibits an anabolic metabolic pathway in a cell.

In some embodiments, the metabolic inhibitor is a PI3K inhibitor, an mTOR inhibitor, an AKT inhibitor, a glucose metabolism inhibitor, a glutamine metabolism inhibitor, an inhibitor of reactive oxygen production, metformin, or a combination thereof.

In some embodiments, the PI3K inhibitor is idelalisib.

In some embodiments, the condition is an autoimmune disease, a chronic infection, or cancer. Other conditions are also contemplated, particularly any condition that is suggested to be treated by administration of anti-PD-1 therapy.

In some embodiments, the autoimmune disease is Chron's disease, inflammatory bowel disease, autoimmune diabetes, or lupus.

In some embodiments, the chronic infection is tuberculosis, malaria, HIV infection, hepatitis B, hepatitis C, cytomegalovirus, or Epstein-Barr virus.

In some embodiments, the cancer is colon cancer, prostate cancer, bladder cancer, soft-tissue sarcoma, an advanced lung cancer, non-small cell lung cancer, small cell lung cancer, mesothelioma, esophageal cancer, renal cell cancer, melanoma, basal cell skin cancer, squamous cell skin cancer, and squamous cell carcinoma of the head and neck. However, other cancer types are also contemplated, particularly any cancer that is suggested to be treated by administration of anti-PD-1 therapy.

In some embodiments, the condition is cancer and the subject is further administered a chemotherapeutic, radiation therapy, anti-CTLA-4, anti-TIM-3, anti-TIGIT, anti-CD40, a TLR agonist, a STING agonist, a cancer vaccine, adoptive T-cell therapy, CAR-T cell therapy, or an anti-myeloid cell therapy.

In some embodiments, the level of TCF1 in the subject is measured from a blood sample.

In some embodiments, the level of TCF1 in the subject is measured from a tumor sample.

According to embodiments of the present invention, there is provided a method of treating a condition in a subject by providing an anti-PD-1 treatment to the subject, comprising the steps of:
a) administering an anti-PD1 treatment to the subject;
b) monitoring the clinical response of the condition in the subject to the anti-PD-1 therapy;
c) identifying reduced anti-PD-1 treatment efficacy in the subject and classifying the subject as having an anti-PD-1 non-responder phenotype if the clinical response decreases; and
d) administering a metabolic inhibitor to a subject identified as having an anti-PD-1 non-responder phenotype.

In some embodiments, the clinical response is determined by the level of TCF1 in the subject over the course of the anti-PD-1 treatment, wherein a decrease in the level of TCF1 in the subject is interpreted as a decrease in clinical response.

In some embodiments, the level of TCF1 is determined by calculating the proportion of TCF1⁺ T cells among T cells of the subject.

In some embodiments, the level of TCF1 is determined by calculating the proportion of TCF1⁺ T cells among CD8⁺ T cells of the subject.

In some embodiments, the level of TCF1 is determined by calculating the proportion of TCF1⁺ T cells among CD44⁺ T cells of the subject.

In some embodiments, the level of TCF1 is determined by calculating the proportion of TCF1⁺ T cells among Ki-67⁺ T cells of the subject.

In some embodiments, the clinical response is further determined by the level of Ki-67 in the subject over the course of the anti-PD-1 treatment, wherein a decrease in the level of Ki-67 in the subject is interpreted as a decrease in clinical response.

In some embodiments, the clinical response is further determined by the level of Ki-67 in the subject over the course of the anti-PD-1 treatment, wherein a lack of a substantial increase in the level of Ki-67 in the subject after administration of an anti-PD-1 treatment is interpreted as a decrease in clinical response.

In some embodiments, the level of Ki-67 is determined by calculating the proportion of Ki-67⁺ T cells among T cells of the subject.

In some embodiments, the level of Ki-67 is determined by calculating the proportion of Ki-67⁺ T cells among CD8⁺ T cells of the subject.

In some embodiments, the level of Ki-67 is determined by calculating the proportion of Ki-67⁺ T cells among CD44⁺ T cells of the subject.

In some embodiments, the subject is classified as having an anti-PD-1 non-responder phenotype if the subject displays a ratio of Ki-67⁺ T cells/Ki-67⁻ cells of about 0.35 or lower.

In some embodiments, the clinical response is further determined by the tumor volume in the subject over the course of the anti-PD-1 treatment, wherein an increase in tumor volume is interpreted as a decrease in clinical response.

In some embodiments, the level of TCF1 is determined by an immunoassay.

In some embodiments, the immunoassay is a flow cytometry assay or an immunohistochemistry assay.

In some embodiments, the administered amount of the metabolic inhibitor is sufficient to increase the level of TCF1 in the subject.

In some embodiments, the metabolic inhibitor is administered prior to or substantially at the same time as a subsequent administration of an anti-PD-1 therapy.

In some embodiments, a subsequent administration of an anti-PD-1 treatment is administered after detection of an increase in level of TCF1 in a subject identified as having an anti-PD-1 non-responder phenotype.

In some embodiments, the metabolic inhibitor is administered to the subject having an anti-PD-1 responder phenotype

In some embodiments, the metabolic inhibitor is a PI3K inhibitor, an mTOR inhibitor, an AKT inhibitor, a glucose metabolism inhibitor, a glutamine metabolism inhibitor, an inhibitor of reactive oxygen production, metformin, or a combination thereof.

In some embodiments, the PI3K inhibitor is idelalisib.

In some embodiments, the condition is an autoimmune disease, a chronic infection, or cancer.

In some embodiments, the autoimmune disease is Chron's disease, inflammatory bowel disease, autoimmune diabetes, or lupus. As indicated above, other conditions are also contemplated, particularly any condition that is suggested to be treated by administration of anti-PD-1 therapy.

In some embodiments, the chronic infection is tuberculosis, malaria, HIV infection, hepatitis B, hepatitis C, cytomegalovirus, or Epstein-Barr virus.

In some embodiments, the cancer is colon cancer, prostate cancer, bladder cancer, soft-tissue sarcoma, an advanced lung cancer, non-small cell lung cancer, small cell lung cancer, mesothelioma, esophageal cancer, renal cell cancer, melanoma, basal cell skin cancer, squamous cell skin cancer, and squamous cell carcinoma of the head and neck. As indicated above, other cancer types are also contemplated, particularly any cancer that is suggested to be treated by administration of anti-PD-1 therapy.

In some embodiments, the condition is cancer and the subject is further administered a chemotherapeutic, radiation therapy, anti-CTLA-4, anti-TIM-3, anti-TIGIT, anti-CD40, a TLR agonist, a STING agonist, a cancer vaccine, adoptive T-cell therapy, CAR-T cell therapy, or an anti-myeloid cell therapy.

In some embodiments, the level of TCF1 in the subject is measured from a blood sample.

In some embodiments, the level of TCF1 in the subject is measured from a tumor sample.

According to embodiments of the present invention, there is provided a method of classifying an anti-PD-1 non-responder phenoytope in a test sample comprising:
a) determining the level of TCF1 in the test sample by measuring the number of TCF1⁺ T cells in the test sample; and
b) identifying the test sample as displaying an anti-PD-1 non-responder phenotype if the measured ratio of TCF1⁺ cells/TCF1⁻ cells is less than about 0.8.

In some embodiments the test sample is identified as displaying an anti-PD-1 non-responder phenotype if the measured ratio of Ki-67⁺ T cells/Ki-67⁻ T cells is less than about 0.35.

In some embodiments, the test sample is a blood sample.

According to embodiments of the present invention, there is provided a method determining whether a subject is likely to benefit from administration of anti-PD-1 therapy, the method comprising:
a) determining the level of TCF1 in a test sample from the subject by measuring the number of TCF1⁺ T cells in the sample; and
b) identifying the subject as likely to benefit from administration of anti-PD-1 therapy if the measured ratio of TCF1⁺ cells/TCF1⁻ cells is greater than about 0.8, or identifying the subject as unlikely to benefit from administration of anti-PD-1 therapy if the measured ratio of TCF1⁺ cells/TCF1⁻ cells is less than about 0.8.

In some embodiments, the subject is likely to benefit from administration of anti-PD-1 therapy if the measured ratio of Ki-67⁺ T cells/Ki-67⁻ T cells is less than about 0.35.

In some embodiments, the test sample is a blood sample.

According to embodiments of the present invention, there is provided a method of monitoring anti-PD-1 cancer treatment efficacy in a subject undergoing anti-PD-1 therapy, comprising the steps of:
a) obtaining a test sample from the subject prior to administration of an anti-PD-1 therapy;
b) determining a baseline ratio of TCF1⁺ T cells to TCF1⁻ T cells within a population of CD8⁺, Ki-67⁺ T cells of the test sample;
c) administering an anti-PD-1 treatment to the subject;
d) obtaining periodic samples from the subject over the course of the anti-PD-1 therapy and determining a ratio of TCF1⁺ T cells to TCF1⁻ T cells within a population of CD8⁺, Ki-67⁺ T cells of each sample; and
e) identifying reduced anti-PD-1 treatment efficacy in the subject and classifying the subject as having an anti-PD-1 non-responder phenotype if ratio of TCF1⁺ T cells to TCF1⁻ T cells within a population of CD8⁺, Ki-67⁺ T cells decreases relative to the baseline ratio.

In some embodiments, the test sample is a blood sample.

According to embodiments of the present invention, there is provided a kit comprising a reagent for measuring the level of TCF1 in a test sample of a subject and instructions for identifying the subject as having an anti-PD-1 responder phenotype or an anti-PD-1 non-responder phenotype based on the measured level of TCF1 in the test sample.

In some embodiments, the reagent is an anti-TCF1 antibody.

In some embodiments, the kit further comprises an anti-Ki-67 antibody.

In some embodiments, the test sample is a blood sample.

According to embodiments of the present invention, there is provided a method determining whether a subject is likely to benefit from administration of anti-PD-1 therapy, the method comprising:
a) determining the level of TCF1 in a test sample from the subject by measuring the % TCF1⁺ T cells among CCR7⁻, CD8⁺ T cells in the test sample; and
b) identifying the subject as likely to benefit from administration of anti-PD-1 therapy if the measured the % TCF1⁺ T cells is greater than about 40%, preferably greater than about 45%, or identifying the subject as unlikely to benefit from administration of anti-PD-1 therapy if the measured the % TCF1⁺ T cells is less than about 25%, preferably less than about 20%.

In some embodiments, the test sample is a blood sample.

According to embodiments of the present invention, there is provided a method of monitoring anti-PD-1 cancer treatment efficacy in a subject undergoing anti-PD-1 therapy, comprising the steps of:
a) obtaining a test sample from the subject prior to administration of an anti-PD-1 therapy;
b) determining a baseline ratio of TCF1⁺ T cells to TCF1⁻ T cells within a population of PD-1⁺, CCR7⁻, CD8⁺ T cells of the test sample;
c) administering an anti-PD-1 treatment to the subject;
d) obtaining periodic samples from the subject over the course of the anti-PD-1 therapy and determining a ratio of TCF1⁺ T cells to TCF1⁻ T cells within a population of PD-1⁺, CCR7⁻, CD8⁺ T cells of each sample; and
e) identifying reduced anti-PD-1 treatment efficacy in the subject and classifying the subject as having an anti-PD-1 non-responder phenotype if ratio of TCF1⁺ T cells to TCF1⁻ T cells within a population of PD-1⁺, CCR7⁻, CD8⁺ T cells decreases relative to the baseline ratio.

In some embodiments, the subject is identified as likely to benefit from administration of anti-PD-1 therapy if the measured ratio of TCF1⁺ cells/TCF1⁻ cells is greater than about 0.8, preferably about 1.5 higher, or identifying the subject as unlikely to benefit from administration of anti-PD-1 therapy if the measured ratio of TCF1⁺ cells/TCF1⁻ cells is less than about 0.8, preferably about 0.6 or lower.

In some embodiments, the test sample is a blood sample.

The present invention describes a strategy for enhancing the effects of immunotherapy treatments for cancer by manipulating metabolic pathways to increase the levels of progenitor, TCF1⁺, T cells in the tumor and peripheral blood prior to anti-PD-1 therapy.

Using several murine models of infection and cancer, the present disclosure demonstrates that antigen-specific T cells regenerate via a metabolically-regulated process that is particularly sensitive to PI3 kinase pathway activation. TCF1⁺ T cells are capable of self-renewal, and loss of TCF1 expression on CD8⁺ T cells occurs when the immune system enters a state of “terminal differentiation” wherein T cells exhibit proliferation and effector function at the expense of durability.

Accordingly, this disclosure describes several clinical applications based on applying the mechanism described above. For example, measurement of TCF1⁺ T cells in a pre-treatment tumor or blood sample serves as a predictor of response to standard immunotherapy. Subjects with low baseline levels of TCF1 may be less likely to experience measurable radiographic response or clinical benefit from anti-PD-1 treatments. The inventors have shown that in murine models, administration of PI3K inhibitors can repopulate TCF1⁺ T cells in a tumor and peripheral blood as compared to mice that are untreated or treated with anti-PD-1 or PI3K inhibitors alone. This combination yields improved tumor control and duration of response to treatment relative to single-agent approaches.

As a non-limiting example, the present technology provides a method of advanced lung cancer treatment by combining PI3K inhibitors with anti-PD-1 therapy in lung cancer patients. Notably, all subjects may benefit from increased TCF-1+ T cell levels prior to anti-PD-1 therapy as well as those subjects with low measured TCF1⁺ T cell levels. Serial tracking of TCF1⁺ T cell levels in the peripheral blood may serve as a surrogate indicator of efficacy while on treatment. Because it is like that immunotherapy response is driven by the balance of self-renewing versus effector T cells and of T cells versus tumor cells, measurement of T cell populations in the peripheral blood during treatment may permit non-continuous dosing to achieve sufficient levels of T cells for clinical response, improving the toxicity profile of combination approaches currently in testing. The ability to achieve increased TCF1⁺ T cells could inform further therapeutic development in other combination approaches. Furthermore, the lack thereof could highlight a previously unrecognized patient population with unmet medical need.

The invention also includes metabolic manipulation via other means (e.g. administration of metformin, AKT inhibitors, and others) to achieve increased self-renewing T cell populations in a subject, for example, a subject that has cancer. The invention also includes metabolic manipulation via these pathways combined with anti-PD-1 axis therapy and other agents, such as chemotherapy, radiation, anti-CTLA-4, anti-TIM-3, anti-TIGIT, anti-CD40, TLR agonists, STING agonists, cancer vaccines, adoptive T cell therapy expansion, CAR T cells, or anti-myeloid cell therapies.

In summary, the inventive technology is a method of enhancing the immune response of a subject and an improved method of monitoring and treating a condition in a subject. The inventive technology provides a mechanistic understanding of a subject's immune response to a therapy, allowing for improved patient selection, therapeutic monitoring, reduced toxicity, and an overall improvement in clinical outcome. More specifically, presented herein is a novel therapeutic approach of utilizing an anti-anabolic agent with immunotherapy to improve durability of a clinical response e.g. tumor response, to the immunotherapy. Also presented is a novel non-invasive, biomarker-based approach for determining an index of T cell regenerative capacity, and an actionable tool for therapy selection based on determined index. Various other inventive aspects can be integrated or employed, as discussed infra.

While the invention has been particularly shown and described with reference to a preferred embodiment and various alternate embodiments, it will be understood by persons skilled in the relevant art that various changes in form and details can be made therein without departing from the spirit and scope of the invention.

### EQUIVALENTS AND INCORPORATION BY REFERENCE

All references cited herein are incorporated by reference to the same extent as if each individual publication, database entry (e.g. Genbank sequences or GeneID entries), patent application, or patent, was specifically and individually indicated to be incorporated by reference in its entirety, for all purposes. This statement of incorporation by reference is intended by Applicants, pursuant to 37 C.F.R. § 1.57(b)(1), to relate to each and every individual publication, database entry (e.g. Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

### EXPERIMENTAL DETAILS

### Materials and Methods:

### Rodent Data Methods:

The following detailed methodology was used to generate the initial rodent data presented herein. For tumor challenge models, C57BL/6 mice were inoculated subcutaneously in the right flank with 2×10⁵ tumor cells (either MC38 carcinoma or MCA205-Ova fibrsocarcoma). Mice were monitored and tumors measured daily across two dimensions. To calculate tumor volumes, the formula: (mm³)=0.52×(length)×(width) was used; 0.52 is an approximation of p/6. Ante-mortem immune monitoring was performed on peripheral blood or fine-needle aspirates from tumor. Post-mortem tumor cell suspensions were prepared by cutting tumors into small pieces (<0.1 g) and then dissociating mechanically and enzymatically. Flow cytometry was performed with antibodies detecting CD8, CD44, Ki67, TCF1 and a tetramer reagent to detect Ova-specific T cells in the MCA205-Ova model.

### Human Blood Flow Cytometry Methods:

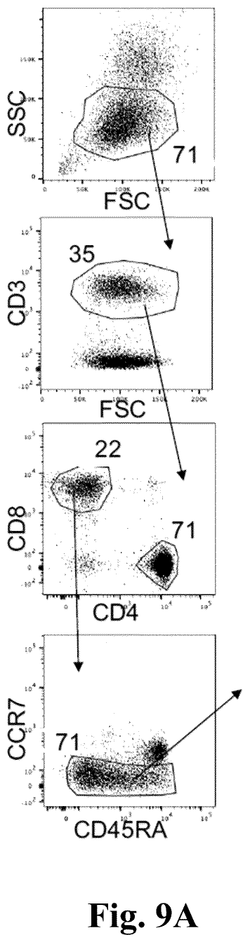

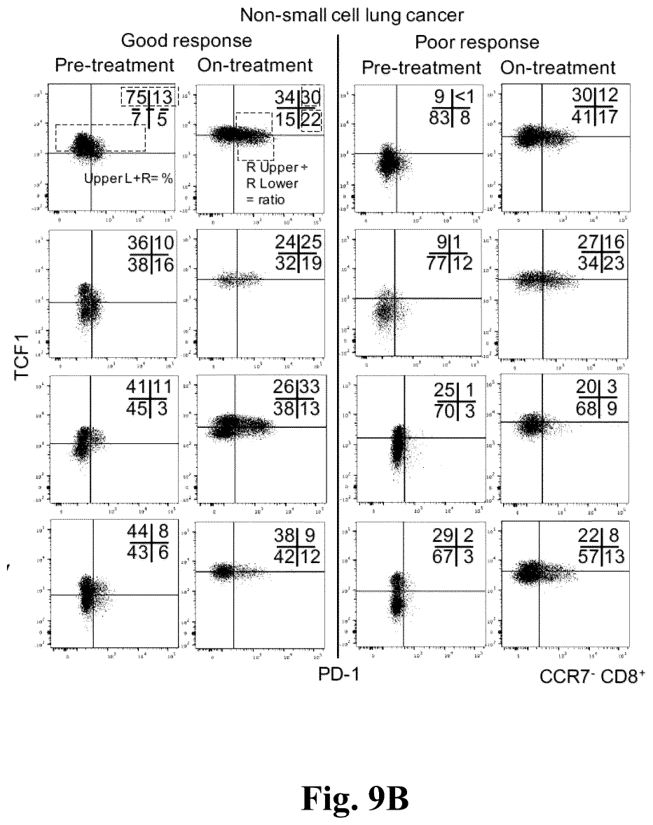

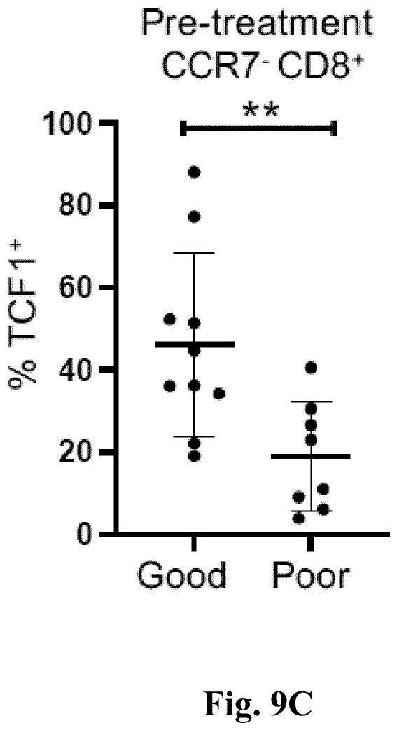

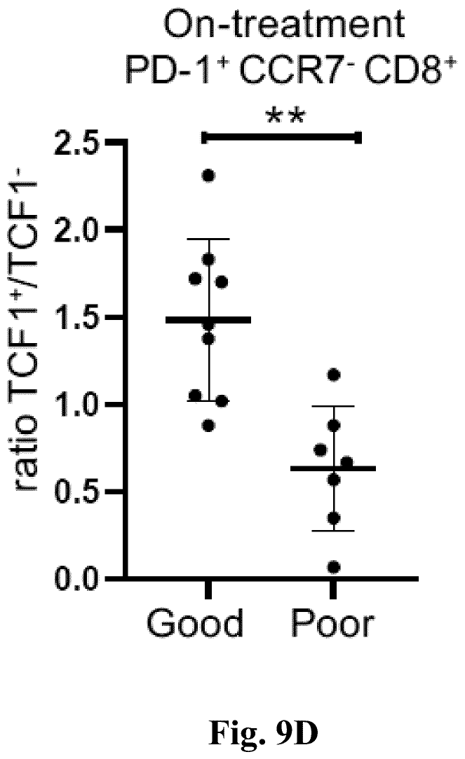

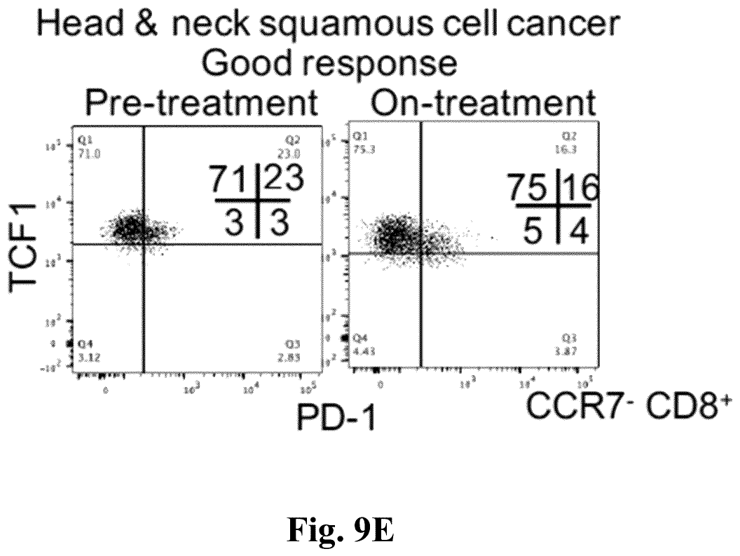

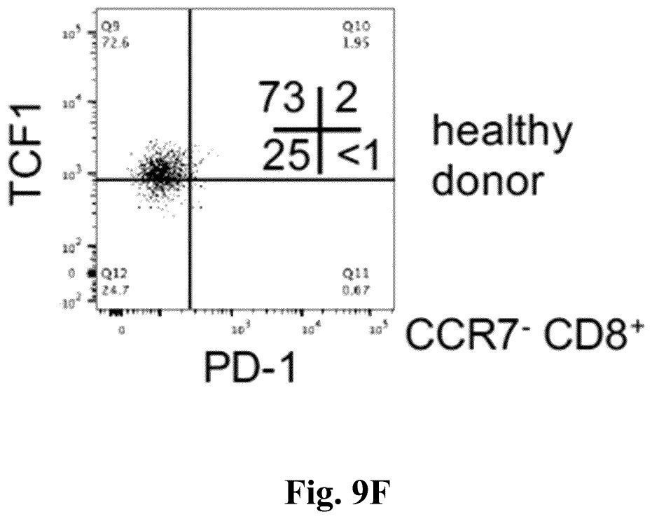

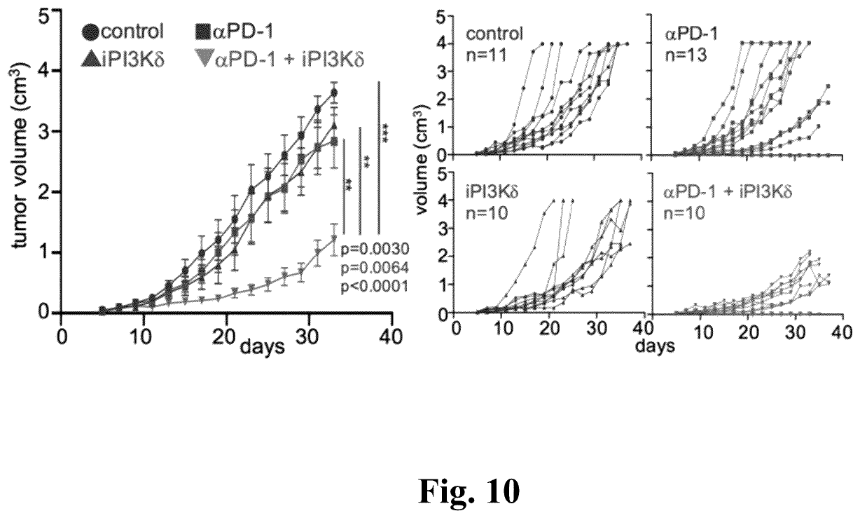

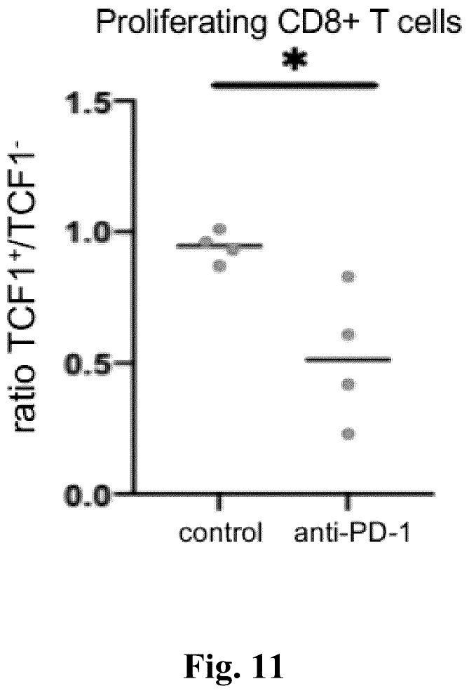

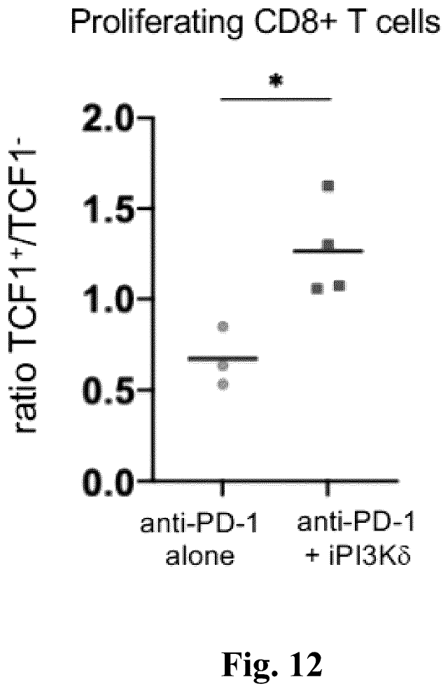

Baseline (pre-treatment) patient testing. Using conventional multiparametric flow cytometry, baseline (pre-treatment) peripheral blood mononuclear cells (PBMCs) from responder and non-responder non-small cell lung cancer (NSCLC) patients treated with immune checkpoint blockade (anti-PD-1) were examined using a panel of commercially-available antibodies against T cell subsets (CD3, CD8, CD4), key transcription factors (TCF1, Eomes, T-bet, FOXP3), proliferation markers (Ki-67), and markers covering the spectrum of self-renewing, effector, and exhausted T cells (CD45RA, CCR7, CD127, CD27, CX3CR1, CD57, CD39, PD-1, CTLA-4). Samples were collected on a Bio-Rad ZE5 Apollo cell analyzer. The pre-treatment samples from the first set of 10 good-responders and 8 poor-responders are shown in FIG. 9C.

Blood CD8⁺ T cells span the spectrum of peripheral T cell renewal and differentiation. Naive and central memory T cells (CCR7⁺) are more quiescent and self-renewing; and are thus enriched in TCF1⁺ cells. The initial focus of our analyses has started with the more differentiated effector memory T cells (CCR7⁻), both those that are CD45RA⁻ and the even-more differentiated CD45RA⁺ subset. Collectively, CCR7⁻ cells have lower proportions of TCF1⁺ cells than do CCR7⁺ cells, but in active immune responses, the frequency of TCF1⁺ cells among CCR7⁻ subsets increases. Initial analyses of the CCR7⁻ CD8⁺ T cells from the baseline samples suggests that patients who respond to immune checkpoint blockade (ICB) are more likely to have higher pre-treatment TCF1⁺ frequency (mean=46%), while a lower TCF1⁺ frequency (mean=19%) in CCR7⁻ CD8⁺ T cells predicts greater likelihood of resistance to anti-PD-1 therapy (**P=0.0076).

The value of this test is to determine non-invasively which patients are candidates to commence immune checkpoint blockade therapy and which are unlikely to respond. It may be the case that patients with lower baseline TCF1⁺ frequency can be treated with metabolic manipulation prior to anti-PD-1 in order to increase the frequency of TCF1⁺ T cells in their CCR7⁻ CD8 blood T cell compartment.

On-treatment patient testing. Using the same flow cytometry panel as used for the pre-treatment samples, the ratio of TCF1⁺/TCF1⁻ cells in selected CD8⁺ T cell subsets were analyzed to see if this predicts durability of the response, and if decay of the TCF1⁺ proportion portends development of resistance to PD-1 blockade. The initial analyses of the CCR7⁻ CD8⁺ T cells from the first 16 on-treatment samples from 9 good-responders and 7 poor-responders has begun. Some non-responder patients exhibit increased frequency of TCF1⁺ T cells after treatment, while others do not show a dramatic increase over baseline. More uniformly, all responders and most non-responders exhibited increased frequency of PD-1⁺ T cells on-treatment compared to baseline samples. Because of the apparent therapy-induced expansion of PD-1⁺ CCR7⁻ CD8⁺ T cells, the on-treatment PBMCs from NSCL patients were examined for the ratio of TCF1⁺/TCF1⁻ cells just among the PD-1⁺, CCR7⁻, CD8⁺ T cells, as shown in FIG. 9D. Patients who respond to immune checkpoint blockade ICB are more likely to have higher on-treatment TCF1⁺/TCF1⁻ ratio (mean=1.5) in the PD-1⁺, CCR7⁻, CD8⁺ pool, while a lower TCF1⁺/TCF1⁻ ratio (mean=0.6) in PD-1⁺, CCR7⁻, CD8⁺ T cells predicts greater likelihood of resistance to anti-PD-1 therapy (**P=0.0013).

The value of this test is to determine non-invasively which patients are likely to develop resistance to immune checkpoint blockade after commencing therapy. Some of the patients with declining ratios of TCF1⁺/TCF1⁻, PD-1⁺, CCR7⁻, CD8⁺ T cells may be candidates for the addition of metabolic manipulation prior to their next rounds of anti-PD-1.

### Quantitative Multiplex Immunofluorescence Methods:

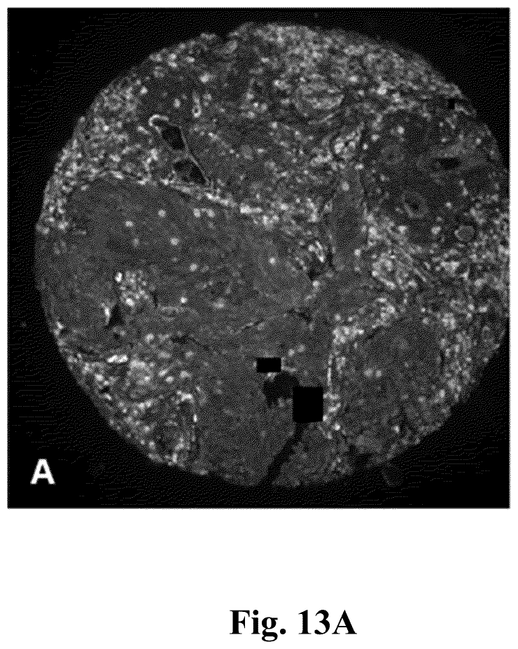

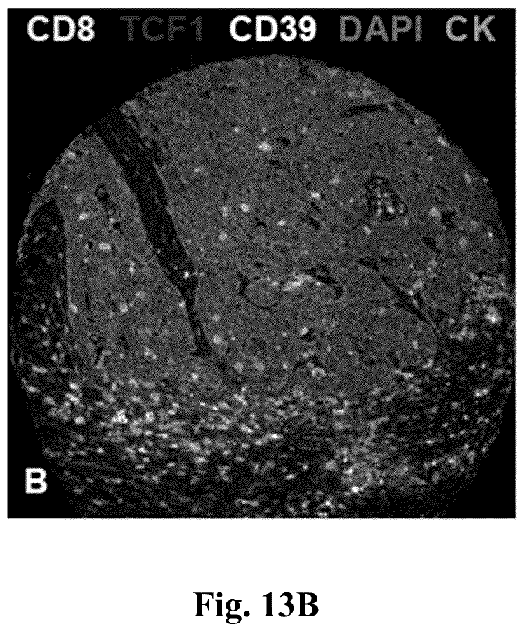

Validating quantitative multiplex immunofluorescence microscopy of human cancer tissue. Markers (with indicated color in FIG. 13B) include CD8, TCF1, CD39, DAPI (DNA dye), and tumor cytokeratin (CK, a lung cancer marker). Two representative tissue cores (FIGS. 13A and 13B) revealing some areas of denser inflammation at the tumor interface containing CD8⁺, TCF1⁺ cells, CD8⁺, TCF1⁻ cells, and some CD39⁺ cells (both TCF1⁺ and TCF1⁻). There are also scattered CD8⁺, TCF1⁻ cells within tumor tissue.

Although the analyses of the pre-treatment biopsies from good-responding and poor-responding patients with non-small cell lung cancer has not yet been performed, it is anticipated that the ratio of TCF1⁺/TCF1⁻ CD8⁺ T cells in pre-treatment biopsies from human patients will provide supportive information of the value of the TCF1⁺ frequency in CD8⁺ T cells from pre-treatment blood as a predictive biomarker for response versus resistance to anti-PD-1 treatment.

Detailed Methodology: Using quantitative multiplex immunofluorescence, we will measure TCF1⁺/TCF1⁻ ratio of CD8⁺ T cells from baseline (pre-treatment) formalin-fixed, paraffin embedded (FFPE) tissue. The panel validation has recently been completed using staining of tissue microarrays of non-small cell lung cancer specimens. The five-color panel includes the tumor marker cytokeratin (CK), CD8, TCF1, CD39, and DAPI to resolve nuclei. The focus of the panel is to distinguish between self-renewing (TCF1⁺, CD39⁻) and irreversibly differentiated or exhausted (TCF1⁻, CD39⁺) CD8⁺ T cells.

Image acquisition and analysis will be conducted using the Vectra platform. The resulting images are digitally processed in order to appropriately separate the overlapping emission spectra of these fluorophores, and the output is a set of digital files of multi-layered images which can be analyzed on subsequent image processing platforms to determine, for example, the spatial relationship of immune cells in tissues. This platform utilizes an automated image analysis software package. The workflow for this software consists of image preparation, tissue segmentation (i.e. tumor versus stroma), cell segmentation, and marker scoring by intensity, allowing for single-cell quantification of immune cells co-expressing markers of interest.

### REFERENCES

Adams, W. C., Chen, Y. H., Kratchmarov, R., Yen, B., Nish, S. A., Lin, W. W., Rothman, N.J., Luchsinger, L. L., Klein, U., Busslinger, M., et al. (2016). Anabolism-Associated Mitochondrial Stasis Driving Lymphocyte Differentiation over Self-Renewal. Cell reports 17, 3142-3152.
Blank, C. U., Haining, W. N., Held, W., Hogan, P. G., Kallies, A., Lugli, E., Lynn, R. C., Philip, M., Rao, A., Restifo, N. P., et al. (2019). Defining ‘T cell exhaustion’. Nature reviews Immunology, 19(11):665-674. doi: 10.1038/s41577-019-0221-9.
Bowers, J. S., Majchrzak, K., Nelson, M. H., Aksoy, B. A., Wyatt, M. M., Smith, A. S., Bailey, S. R., Neal, L. R., Hammerbacher, J. E., and Paulos, C. M. (2017). PI3Kdelta Inhibition Enhances the Antitumor Fitness of Adoptively Transferred CD8(+) T Cells. Frontiers in immunology 8, 1221.
Chen, Y. H., Kratchmarov, R., Lin, W. W., Rothman, N.J., Yen, B., Adams, W. C., Nish, S. A., Rathmell, J. C., and Reiner, S. L. (2018). Asymmetric PI3K Activity in Lymphocytes Organized by a PI3K-Mediated Polarity Pathway. Cell reports 22, 860-868.
Huang, A. C., Postow, M. A., Orlowski, R. J., Mick, R., Bengsch, B., Manne, S., Xu, W., Harmon, S., Giles, J. R., Wenz, B., et al. (2017). T-cell invigoration to tumour burden ratio associated with anti-PD-1 response. Nature 545, 60-65.
Huang, A. C., Orlowski, R. J., Xu, X., Mick, R., George, S. M., Yan, P. K., Manne, S., Kraya, A. A., Wubbenhorst, B., Dorfman, L., et al. (2019). A single dose of neoadjuvant PD-1 blockade predicts clinical outcomes in resectable melanoma. Nature medicine 25, 454-461.
Im, S. J., Hashimoto, M., Gerner, M. Y., Lee, J., Kissick, H. T., Burger, M. C., Shan, Q., Hale, J. S., Lee, J., Nasti, T. H., et al. (2016). Defining CD8⁺ T cells that provide the proliferative burst after PD-1 therapy. Nature 537, 417-421.
Kamphorst, A. O., Pillai, R. N., Yang, S., Nasti, T. H., Akondy, R. S., Wieland, A., Sica, G. L., Yu, K., Koenig, L., Patel, N. T., et al. (2017). Proliferation of PD-1+ CD8 T cells in peripheral blood after PD-1-targeted therapy in lung cancer patients. Proceedings of the National Academy of Sciences of the United States of America 114, 4993-4998.
Kratchmarov, R., Magun, A. M., and Reiner, S. L. (2018). TCF1 expression marks self-renewing human CD8(+) T cells. Blood advances 2, 1685-1690.
Lin, W. H., Adams, W. C., Nish, S. A., Chen, Y. H., Yen, B., Rothman, N.J., Kratchmarov, R., Okada, T., Klein, U., and Reiner, S. L. (2015). Asymmetric PI3K signaling driving developmental and regenerative cell fate bifurcation. Cell reports 13, 2203-2218.
Lin, W. W., Nish, S. A., Yen, B., Chen, Y. H., Adams, W. C., Kratchmarov, R., Rothman, N.J., Bhandoola, A., Xue, H. H., and Reiner, S. L. (2016). CD8⁺ T Lymphocyte Self-Renewal during Effector Cell Determination. Cell reports 17, 1773-1782.
Lu, X., Homer, J. W., Paul, E., Shang, X., Troncoso, P., Deng, P., Jiang, S., Chang, Q., Spring, D. J., Sharma, P., et al. (2017). Effective combinatorial immunotherapy for castration-resistant prostate cancer. Nature 543, 728-732.
Nish, S. A., Zens, K. D., Kratchmarov, R., Lin, W. W., Adams, W. C., Chen, Y. H., Yen, B., Rothman, N.J., Bhandoola, A., Xue, H. H., et al. (2017). CD4⁺ T cell effector commitment coupled to self-renewal by asymmetric cell divisions. The Journal of experimental medicine 214, 39-47.
Philip, M., Fairchild, L., Sun, L., Horste, E. L., Camara, S., Shakiba, M., Scott, A. C., Viale, A., Lauer, P., Merghoub, T., et al. (2017). Chromatin states define tumour-specific T cell dysfunction and reprogramming. Nature 545, 452-456.
Sade-Feldman, M., Yizhak, K., Bjorgaard, S. L., Ray, J. P., de Boer, C. G., Jenkins, R. W., Lieb, D. J., Chen, J. H., Frederick, D. T., Barzily-Rokni, M., et al. (2018). Defining T Cell States Associated with Response to Checkpoint Immunotherapy in Melanoma. Cell 175, 998-1013 e1020.
Utzschneider, D. T., Charmoy, M., Chennupati, V., Pousse, L., Ferreira, D. P., Calderon-Copete, S., Danilo, M., Alfei, F., Hofmann, M., Wieland, D., et al. (2016). T Cell Factor 1-Expressing Memory-like CD8(+) T Cells Sustain the Immune Response to Chronic Viral Infections. Immunity 45, 415-427.
Wu, T., Ji, Y., Moseman, E. A., Xu, H. C., Manglani, M., Kirby, M., Anderson, S. M., Handon, R., Kenyon, E., Elkahloun, A., et al. (2016). The TCF1⁻Bcl6 axis counteracts type I interferon to repress exhaustion and maintain T cell stemness. Science Immunology 1(6):eaai8593. doi:10.1126/sciimmunol.aai8593.

What is claimed is:

1. A method of treating a condition in a subject by improving the immune response of the subject, the method comprising:
a) determining the level of TCF1 in the subject to identify the subject as having an anti-PD-1 responder phenotype or an anti-PD-1 non-responder phenotype; and
b) administering (i) an anti-PD-1 treatment to a subject having an anti-PD-1 responder phenotype; or (ii) a metabolic inhibitor to a subject having an anti-PD-1 non-responder phenotype.

2. The method of claim 1, wherein the level of TCF1 is determined by calculating the proportion of TCF1⁺ T cells among T cells of the subject.

3. The method of claim 1 or 2, wherein the level of TCF1 is determined by calculating the proportion of TCF1⁺ T cells among CD8⁺ T cells of the subject.

4. The method of claim 1, 2 or 3, wherein the level of TCF1 is determined by calculating the proportion of TCF1⁺ T cells among CD44⁺ T cells of the subject.

5. The method of claim 1, 2, 3 or 4, wherein the level of TCF1 is determined by calculating the proportion of TCF1⁺ T cells among Ki-67⁺ T cells of the subject.

6. The method of any one of claims 1-5, wherein the subject is identified as having an anti-PD-1 non-responder phenotype if the subject displays a ratio of TCF1⁺ T cells/TCF1⁻ T cells of about 0.8 or lower.

7. The method any one of claims 1-6, further comprising determining the level of Ki-67 in the subject to identify the subject as having an anti-PD-1 responder phenotype or an anti-PD-1 non-responder phenotype.

8. The method of claim 7, wherein the level of Ki-67 is determined by calculating the proportion of Ki-67⁺ T cells among T cells of the subject.

9. The method of claim 7 or 8, wherein the level of Ki-67 is determined by calculating the proportion of Ki-67⁺ T cells among CD8⁺ T cells of the subject.

10. The method of claim 7, 8 or 9, wherein the level of Ki-67 is determined by calculating the proportion of Ki-67⁺ T cells among CD44⁺ T cells of the subject.

11. The method of any one of claims 1-10, wherein the subject is identified as having an anti-PD-1 non-responder phenotype if the subject displays a ratio of Ki-67⁺ T cells/Ki-67⁻ T cells of about 0.35.

12. The method of any one of claims 1-11, further comprising determining the tumor volume in the subject to identify the subject as having an anti-PD-1 responder phenotype or an anti-PD-1 non-responder phenotype.

13. The method of any one of claims 1-12, wherein the level of TCF1 is determined by an immunoassay.

14. The method of claim 13, wherein the immunoassay is a flow cytometry assay or an immunohistochemistry assay.

15. The method of any one of claims 1-14, wherein the administered amount of the metabolic inhibitor is sufficient to increase the level of TCF1 in the subject.

16. The method of any one of claims 1-15, wherein an anti-PD-1 treatment is administered to the subject identified as having an anti-PD-1 non-responder phenotype simultaneously or substantially simultaneously as the metabolic inhibitor.

17. The method of any one of claims 1-15, wherein an anti-PD-1 treatment is administered to the subject identified as having an anti-PD-1 non-responder phenotype after detection of an increase in level of TCF1 in the subject.

18. The method of any one of claims 1-15, wherein the metabolic inhibitor is administered to the subject having an anti-PD-1 responder phenotype.

19. The method of claim 18, wherein the metabolic inhibitor is administered to the subject having an anti-PD-1 responder phenotype simultaneously or substantially simultaneously as the anti-PD-1 treatment.

20. The method of claim 18, wherein the metabolic inhibitor is administered to the subject having an anti-PD-1 responder phenotype after the anti-PD-1 treatment.

21. The method of any one of claims 1-20, wherein the metabolic inhibitor is an inhibitor of proliferative anabolic metabolism.

22. The method of any one of claims 1-20, wherein the metabolic inhibitor is a PI3K inhibitor, an mTOR inhibitor, an AKT inhibitor, a glucose metabolism inhibitor, a glutamine metabolism inhibitor, an inhibitor of reactive oxygen production, metformin, or a combination thereof.

23. The method of claim 22, wherein the PI3K inhibitor is idelalisib.

24. The method of any one of claims 1-23, wherein the condition is an autoimmune disease, a chronic infection, or cancer.

25. The method of claim 24, wherein the autoimmune disease is Chron's disease, inflammatory bowel disease, autoimmune diabetes, or lupus.

26. The method of claim 24, wherein the chronic infection is tuberculosis, malaria, HIV infection, hepatitis B, hepatitis C, cytomegalovirus, or Epstein-Barr virus.

27. The method of claim 24, wherein the cancer is colon cancer, prostate cancer, bladder cancer, soft-tissue sarcoma, an advanced lung cancer, non-small cell lung cancer, small cell lung cancer, mesothelioma, esophageal cancer, renal cell cancer, melanoma, basal cell skin cancer, squamous cell skin cancer, and squamous cell carcinoma of the head and neck.

28. The method of any one of claims 1-27, wherein the condition is cancer and the subject is further administered a chemotherapeutic, radiation therapy, anti-CTLA-4, anti-TIM-3, anti-TIGIT, anti-CD40, a TLR agonist, a STING agonist, a cancer vaccine, adoptive T-cell therapy, CAR-T cell therapy, or an anti-myeloid cell therapy.

29. The method of any one of claims 1-28, wherein the level of TCF1 in the subject is measured from a blood sample.

30. The method of any one of claims 1-28, wherein the level of TCF1 in the subject is measured from a tumor sample.

31. A method of treating a condition in a subject by providing an anti-PD-1 treatment to the subject, comprising the steps of:
a) administering an anti-PD-1 treatment to the subject;
b) monitoring the clinical response of the condition in the subject to the anti-PD-1 therapy;
c) identifying reduced anti-PD-1 treatment efficacy in the subject and classifying the subject as having an anti-PD-1 non-responder phenotype if the clinical response decreases; and
d) administering a metabolic inhibitor to a subject identified as having an anti-PD-1 non-responder phenotype.

32. The method of claim 31, wherein the clinical response is determined by the level of TCF1 in the subject over the course of the anti-PD-1 treatment, wherein a decrease in the level of TCF1 in the subject is interpreted as a decrease in clinical response.

33. The method of claim 32, wherein the level of TCF1 is determined by calculating the proportion of TCF1⁺ T cells among T cells of the subject.

34. The method of claim 31 or 32, wherein the level of TCF1 is determined by calculating the proportion of TCF1⁺ T cells among CD8⁺ T cells of the subject.

35. The method of any one of claims 31-34, wherein the level of TCF1 is determined by calculating the proportion of TCF1⁺ T cells among CD44⁺ T cells of the subject.

36. The method of any one of claims 31-34, wherein the level of TCF1 is determined by calculating the proportion of TCF1⁺ T cells among Ki-67⁺ T cells of the subject.

37. The method of any one of claims 31-36, wherein the subject is classified as having an anti-PD-1 non-responder phenotype if the subject displays a ratio of TCF1⁺ T cells/TCF1⁻ T cells of about 0.8 or lower.

38. The method of any one of claims 31-37, wherein the clinical response is further determined by the level of Ki-67 in the subject over the course of the anti-PD-1 treatment, wherein a decrease in the level of Ki-67 in the subject is interpreted as a decrease in clinical response.

39. The method of any one of claims 31-37, wherein the clinical response is further determined by the level of Ki-67 in the subject over the course of the anti-PD-1 treatment, wherein a lack of a substantial increase in the level of Ki-67 in the subject after administration of an anti-PD-1 treatment is interpreted as a decrease in clinical response.

40. The method of claim 37, 38, or 39, wherein the level of Ki-67 is determined by calculating the proportion of Ki-67⁺ T cells among T cells of the subject.

41. The method of any one of claims 37-40, wherein the level of Ki-67 is determined by calculating the proportion of Ki-67⁺ T cells among CD8⁺ T cells of the subject.

42. The method of any one of claims 37-41, wherein the level of Ki-67 is determined by calculating the proportion of Ki-67⁺ T cells among CD44⁺ T cells of the subject.

43. The method of any one of claims 37-42, wherein the subject is classified as having an anti-PD-1 non-responder phenotype if the subject displays a ratio of Ki-67⁺ T cells/Ki-67⁻ T cells of about 0.35 or lower.

44. The method of any one of claims 31-43, wherein the clinical response is further determined by the tumor volume in the subject over the course of the anti-PD-1 treatment, wherein an increase in tumor volume is interpreted as a decrease in clinical response.

45. The method of any one of claims 31-44, wherein the level of TCF1 is determined by an immunoassay.

46. The method of claim 45, wherein the immunoassay is a flow cytometry assay or an immunohistochemistry assay.

47. The method of any one of claims 31-46, wherein the administered amount of the metabolic inhibitor is sufficient to increase the level of TCF1 in the subject.

48. The method of any one of claims 31-47, wherein the metabolic inhibitor is administered prior to or substantially at the same time as a subsequent administration of an anti-PD-1 therapy.

49. The method of any one of claims 31-47, wherein a subsequent administration of an anti-PD-1 treatment is administered after detection of an increase in level of TCF1 in a subject identified as having an anti-PD-1 non-responder phenotype.

50. The method of any one of claims 31-49, wherein the metabolic inhibitor is an inhibitor of proliferative anabolic metabolism.

51. The method of any one of claims 31-50, wherein the metabolic inhibitor is a PI3K inhibitor, an mTOR inhibitor, an AKT inhibitor, a glucose metabolism inhibitor, a glutamine metabolism inhibitor, an inhibitor of reactive oxygen production, metformin, or a combination thereof.

52. The method of claim 51, wherein the PI3K inhibitor is idelalisib.

53. The method of any one of claims 31-52, wherein the condition is an autoimmune disease, a chronic infection, or cancer.

54. The method of claim 53, wherein the autoimmune disease is Chron's disease, inflammatory bowel disease, autoimmune diabetes, or lupus.

55. The method of claim 53, wherein the chronic infection is tuberculosis, malaria, HIV infection, hepatitis B, hepatitis C, cytomegalovirus, or Epstein-Barr virus.

56. The method of claim 53, wherein the cancer is colon cancer, prostate cancer, bladder cancer, soft-tissue sarcoma, an advanced lung cancer, non-small cell lung cancer, small cell lung cancer, mesothelioma, esophageal cancer, renal cell cancer, melanoma, basal cell skin cancer, squamous cell skin cancer, and squamous cell carcinoma of the head and neck.

57. The method of any one of claims 31-53, wherein the condition is cancer and the subject is further administered a chemotherapeutic, radiation therapy, anti-CTLA-4, anti-TIM-3, anti-TIGIT, anti-CD40, a TLR agonist, a STING agonist, a cancer vaccine, adoptive T-cell therapy, CAR-T cell therapy, or an anti-myeloid cell therapy.

58. The method of any one of claims 31-57, wherein the level of TCF1 in the subject is measured from a blood sample.

59. The method of any one of claims 31-57, wherein the level of TCF1 in the subject is measured from a tumor sample.

60. A method of classifying an anti-PD-1 non-responder phenotype in a test sample from a subject comprising:
a) determining the level of TCF1 in the test sample by measuring the number of TCF1⁺ T cells in the test sample; and
b) identifying the test sample as displaying an anti-PD-1 non-responder phenotype if the measured ratio of TCF1⁺ cells/TCF1⁻ cells is less than about 0.8.

61. The method of claim 60, wherein the test sample is identified as displaying an anti-PD-1 non-responder phenotype if the measured ratio of Ki-67⁺ T cells/Ki-67⁻ T cells is less than about 0.35.

62. The method of claim 60 or 61, wherein the test sample is a blood sample.

63. A method determining whether a subject is likely to benefit from administration of anti-PD-1 therapy, the method comprising:
a) determining the level of TCF1 in a test sample from the subject by measuring the number of TCF1⁺ T cells in the test sample; and
b) identifying the subject as likely to benefit from administration of anti-PD-1 therapy if the measured ratio of TCF1⁺ cells/TCF1⁻ cells is greater than about or identifying the subject as unlikely to benefit from administration of anti-PD-1 therapy if the measured ratio of TCF1⁺ cells/TCF1⁻ cells is less than about 0.8.

64. The method of claim 63, wherein the subject is likely to benefit from administration of anti-PD-1 therapy if the measured ratio of Ki-67⁺ T cells/Ki-67⁻ T cells is less than about 0.35.

65. The method of claim 63 or 64, wherein the test sample is a blood sample.

66. A method of monitoring anti-PD-1 cancer treatment efficacy in a subject undergoing anti-PD-1 therapy, comprising the steps of:
a) obtaining a test sample from the subject prior to administration of an anti-PD-1 therapy;
b) determining a baseline ratio of TCF1⁺ T cells to TCF1⁻ T cells within a population of CD8⁺, Ki-67⁺ T cells of the test sample;
c) administering an anti-PD-1 treatment to the subject;
d) obtaining periodic samples from the subject over the course of the anti-PD-1 therapy and determining a ratio of TCF1⁺ T cells to TCF1⁻ T cells within a population of CD8⁺, Ki-67⁺ T cells of each sample; and
e) identifying reduced anti-PD-1 treatment efficacy in the subject and classifying the subject as having an anti-PD-1 non-responder phenotype if ratio of TCF1⁺ T cells to TCF1⁻ T cells within a population of CD8⁺, Ki-67⁺ T cells decreases relative to the baseline ratio.

67. The method of claim 66, wherein the test sample is a blood sample.

68. A kit comprising a reagent for measuring the level of TCF1 in a test sample of a subject and instructions for identifying the subject as having an anti-PD-1 responder phenotype or an anti-PD-1 non-responder phenotype based on the measured level of TCF1 in the test sample.

69. The kit of claim 68, wherein the reagent is an anti-TCF1 antibody.

70. The kit of claim 69, further comprising an anti-Ki-67 antibody.

71. The kit of any one of claims 68-70, wherein the test sample is a blood sample.

72. A method determining whether a subject is likely to benefit from administration of anti-PD-1 therapy, the method comprising:
a) determining the level of TCF1 in a test sample from the subject by measuring the % TCF1⁺ T cells among CCR7⁻, CD8⁺ T cells in the test sample; and
b) identifying the subject as likely to benefit from administration of anti-PD-1 therapy if the measured the % TCF1⁺ T cells is greater than about 40%, preferably greater than about 45%, or identifying the subject as unlikely to benefit from administration of anti-PD-1 therapy if the measured the % TCF1⁺ T cells is less than about 25%, preferably less than about 20%.

73. The method of claim 72, wherein the test sample is a blood sample.

74. A method of monitoring anti-PD-1 cancer treatment efficacy in a subject undergoing anti-PD-1 therapy, comprising the steps of:
a) obtaining a test sample from the subject prior to administration of an anti-PD-1 therapy;
b) determining a baseline ratio of TCF1⁺ T cells to TCF1⁻ T cells within a population of PD-1⁺, CCR7⁻, CD8⁺ T cells of the test sample;
c) administering an anti-PD-1 treatment to the subject;
d) obtaining periodic samples from the subject over the course of the anti-PD-1 therapy and determining a ratio of TCF1⁺ T cells to TCF1⁻ T cells within a population of PD-1⁺, CCR7⁻, CD8⁺ T cells of each sample; and
e) identifying reduced anti-PD-1 treatment efficacy in the subject and classifying the subject as having an anti-PD-1 non-responder phenotype if ratio of TCF1⁺ T cells to TCF1⁻ T cells within a population of PD-1⁺, CCR7⁻, CD8⁺ T cells decreases relative to the baseline ratio.

75. The method of claim 74 wherein the subject is identified as likely to benefit from administration of anti-PD-1 therapy if the measured ratio of TCF1⁺ cells/TCF1⁻ cells is greater than about 0.8, preferably about 1.5 higher, or identifying the subject as unlikely to benefit from administration of anti-PD-1 therapy if the measured ratio of TCF1⁺ cells/TCF1⁻ cells is less than about 0.8, preferably about 0.6 or lower.

76. The method of claim 74 or 75, wherein the test sample is a blood sample.
